(19) United States
(12) Patent Application Publication
(10) Pub. No.: US 2024/0242827 A1
(43) Pub. Date: Jul. 18, 2024

(54) ELECTRONIC DEVICE FOR GENERATING MULTI-PERSONA AND OPERATION METHOD OF THE SAME

(71) Applicant: ELECTRONICS AND TELECOMMUNICATIONS RESEARCH INSTITUTE, Daejeon (KR)

(72) Inventors: SEUNG EUN CHUNG, Daejeon (KR); Kyoung Ju NOH, Daejeon (KR); Jeong Muk LIM, Daejeon (KR); Hyun Tae JEONG, Daejeon (KR)

(21) Appl. No.: 18/497,211

(22) Filed: Oct. 30, 2023

(30) Foreign Application Priority Data
Jan. 13, 2023 (KR) 10-2023-0005136

(51) Int. Cl. G16H 40/63

(52) U.S. Cl. CPC G16H 40/63

(57) ABSTRACT

Disclosed is a user device, which includes a plurality of sensors that collects sensing data, a data analysis device that receives the sensing data from a first server or the plurality of sensors to generate personal characteristic data, and a data storage device that stores the personal characteristic data as default parameters, and the data storage device provides the default parameters and the personal characteristic data to a user through an interface, and the data storage device outputs parameters and the personal characteristic data to a second server based on a user input.

### CROSS-REFERENCE TO RELATED APPLICATION

This application claims priority under 35 U.S.C. § 119 to Korean Patent Application No. 10-2023-0005136 filed on Jan. 13, 2023, in the Korean Intellectual Property Office, the disclosures of which are incorporated by reference herein in their entireties.

### BACKGROUND

### 1. Field of the Invention

Embodiments of the present disclosure described herein relate to an electronic device, and more particularly, relate to an electronic device that generates a multi-persona used in various metaverse environments based on sensing data and a method of operating the same.

### 2. Description of Related Art

A metaverse environment, which refers to the combination of physical reality and the digital world, is evolving toward building an immersive virtual world where the boundaries between the real and digital realms disappear. Creating a metaverse persona, which is named by various names such as a metaverse avatar, virtual persona, or digital persona, may be necessary for users trying new experiences in various metaverse environments.

The metaverse persona may be created as ideal beings based on imagination, or may be created as digital versions with similar tendencies to actual users. Companies providing metaverse services are introducing various methods to produce and provide hyperreal avatars, synthesized voices, and content used in the metaverse environment.

Existing technologies for creating virtual digital avatars include additional user selection of game skins, items, costumes, voices, speech patterns, etc., such that user's choice and personal preference for one of the predefined characters can be reflected as an example. However, technologies that forms a virtual digital avatar by selecting predefined options may not sufficiently reflect the user's characteristics and tastes and may be less immersive.

In addition, since existing technologies for forming virtual digital avatars limit various individual characteristics to a few categories, the digital avatar may lack realism or may be inconsistent and discontinuous when conflicting options are selected.

### SUMMARY

Embodiments of the present disclosure provide an electronic device that generates a multi-persona based on personal characteristic data generated through a result of analysis of sensing data and a method of operating the same.

According to an embodiment of the present disclosure, a user device includes a plurality of sensors that collects sensing data, a data analysis device that receives the sensing data from a first server or the plurality of sensors to generate personal characteristic data, and a data storage device that stores the personal characteristic data as default parameters, and the data storage device provides the default parameters and the personal characteristic data to a user through an interface, and the data storage device outputs parameters and the personal characteristic data to a second server based on a user input.

According to an embodiment, the personal characteristic data may include information about at least one of personality and disposition of the user, feelings and emotional patterns of the user, and life patterns of the user.

According to an embodiment, the data analysis device may receive the sensing data in synchronization with a pre-learning model included in the first server and may generate the personal characteristic data.

According to an embodiment, the pre-learning model may be trained to group each of a plurality of personal characteristics by receiving users' sensing information from the data storage device before the data analysis device receives the sensing data, and when the sensing data is received, the data analysis device may output a personal characteristic corresponding to the sensing data among the plurality of grouped personal characteristics, and may retrain the pre-learning model.

According to an embodiment, the data analysis device may output first personal characteristic data including a first persona based on the sensing data, and the data analysis device may output second personal characteristic data including a second persona based on the sensing data.

According to an embodiment, the data storage device may store the first personal characteristic data and the second personal characteristic data as the default parameters, the second server may include a first metaverse environment and a second metaverse environment, a parameter associated with the first personal characteristic data among the parameters may be output to the first metaverse environment as the first persona, and a parameter associated with the second personal characteristic data among the parameters may be output to the second metaverse environment as the second persona.

According to an embodiment, the sensing data may include first data stored in the data storage device in advance or received from the first server, and second data collected in real time from the plurality of sensors, and the data analysis device may receive the first data and the second data by varying a first weight of the first data and a second weight of the second data.

According to an embodiment, when a capacity of the first data is less than a capacity of the second data, and a comparison value of the first data and the second data is greater than a threshold value, the data analysis device may receive the first data by decreasing the first weight, and the data analysis device may receive the second data by increasing the second weight.

According to an embodiment, when a capacity of the first data is greater than a capacity of the second data, the data analysis device may decrease or increase the first weight depending on whether a data trust value of the first data exceeds a threshold value.

According to an embodiment, when a capacity of the first data is less than a capacity of the second data, and a comparison value of the first data and the second data is less than or equal to a threshold value, the data analysis device may decrease or increase the first weight depending on whether a data trust value of the first data exceeds a threshold value.

According to an embodiment, the sensing data may include first data including information on a behavior intensity of the user for each unit of time, and second data including information on an app use degree of the user for the each unit of time, and the data analysis device may receive the first data and the second data as one vector based on a correlation between the first data and the second data.

According to an embodiment, the sensing data may include first to n-th time series data, and the data analysis device may perform a first analysis on the first to n-th time series data for a first time, the data analysis device may perform a second analysis on the first to n-th time series data for a second time after the first time, and the data analysis device may perform a third analysis on the first to n-th time series data for a third time after the second time, and the personal characteristic data may be generated based on at least one of the first to n-th time series data analyzed during the third time, the second analysis may be performed based on a result of the first analysis, the third analysis may be performed based on a result of the second analysis, the second time may be longer than the first time, and the third time may be longer than the second time.

According to an embodiment of the present disclosure, a method of operating an electronic device including a user device and a first server, includes collecting, by the user device, sensing data, generating, by the first server, personal characteristic data based on the sensing data received from the user device, storing, by the first server, the personal characteristics data as default parameters, providing, by the user device, the default parameters and the personal characteristic data to a user through an interface, and outputting, by the user device, parameters and the personal characteristic data to a second server based on a user input.

According to an embodiment, the generating of the personal characteristic data may include analyzing, by the user device, the sensing data in synchronization with a pre-learning model included in the first server.

According to an embodiment, the pre-learning model may be trained to group each of a plurality of personal characteristics by receiving users' sensing information before analyzing the sensing data, and the analyzing of the sensing data may include outputting, by the user device, a personal characteristic corresponding to the sensing data among the plurality of grouped personal characteristics.

According to an embodiment, the outputting of the personal characteristic may include outputting, by the user device, first personal characteristic data including a first persona based on the sensing data, and outputting, by the user device, second personal characteristic data including a second persona based on the sensing data.

According to an embodiment, the second server may include a first metaverse environment and a second metaverse environment, and the method may further include storing, by the user device, the first personal characteristic data and the second personal characteristic data as the default parameters, outputting, by the user device, a parameter associated with the first personal characteristic data among the parameters to the first metaverse environment as the first persona, and outputting, by the user device, a parameter associated with the second personal characteristic data among the parameters to the second metaverse environment as the second persona.

According to an embodiment, the sensing data may include first data stored in the user device in advance or received from the first server, and second data collected by the user device in real time, and the generating of the personal characteristic data may include synchronizing, by the user device, with a pre-learning model included in the first server, and analyzing, by the user device, the first data and the second data by varying a first weight of the first data and a second weight of the second data.

According to an embodiment, the sensing data may include first data including information on a behavior intensity of the user for each unit of time, and second data including information on an app use degree of the user for the each unit of time, and the generating of the personal characteristic data may include analyzing, by the user device, the first data and the second data as one vector based on a correlation between the first data and the second data.

According to an embodiment, the sensing data may include first to n-th time series data, and the generating of the personal characteristic data may include performing, by the user device, a first analysis on the first to n-th time series data for a first time, performing, by the user device, a second analysis on the first to n-th time series data for a second time after the first time, and performing, by the user device, a third analysis on the first to n-th time series data for a third time after the second time, and the personal characteristic data may be generated based on at least one of the first to n-th time series data analyzed during the third time, the second analysis may be performed based on a result of the first analysis, the third analysis may be performed based on a result of the second analysis, the second time may be longer than the first time, and the third time may be longer than the second time.

### BRIEF DESCRIPTION OF THE DRAWINGS

The above and other objects and features of the present disclosure will become apparent by describing in detail embodiments thereof with reference to the accompanying drawings.

FIG. 1 is a diagram illustrating an electronic device for generating a multi-persona, according to an embodiment of the present disclosure.

FIG. 2 is a block diagram illustrating how a data analysis device utilizes sensing data.

FIG. 3 is a flowchart for determining sensing data received by a data analysis device.

FIG. 4A is a diagram related to a first embodiment of a life pattern analysis result generated by a data analysis device.

FIG. 4B is a diagram related to a second embodiment of a life pattern analysis result generated by a data analysis device.

FIG. 4C is a diagram related to a third embodiment of a life pattern analysis result generated by a data analysis device.

FIG. 5A is a diagram related to a first embodiment illustrating a correlation between various sensing data.

FIG. 5B is a diagram related to a second embodiment illustrating a correlation between various sensing data.

FIG. 6 is a block diagram illustrating how a pre-learning model analyzes sensing data and groups personal characteristic data.

FIG. 7 is a block diagram illustrating how a pre-learning model performs hierarchical unit time analysis on sensing data and outputs personal characteristics.

FIG. 8A is a diagram related to a first embodiment of personal characteristic settings provided by a user device.

FIG. 8B is a diagram related to a second embodiment of personal characteristic settings provided by a user device.

FIG. 8C is a diagram related to a third embodiment of personal characteristic settings provided by a user device.

FIG. 8D is a diagram related to a fourth embodiment of personal characteristic settings provided by a user device.

FIG. 8E is a diagram related to a fifth embodiment of personal characteristic settings provided by a user device.

FIG. 9 is a flowchart of an operation method for generating a multi-persona by an electronic device, according to the present disclosure.

### DETAILED DESCRIPTION

Hereinafter, embodiments of the present disclosure may be described in detail and clearly to such an extent that an ordinary one in the art easily implements the present disclosure.

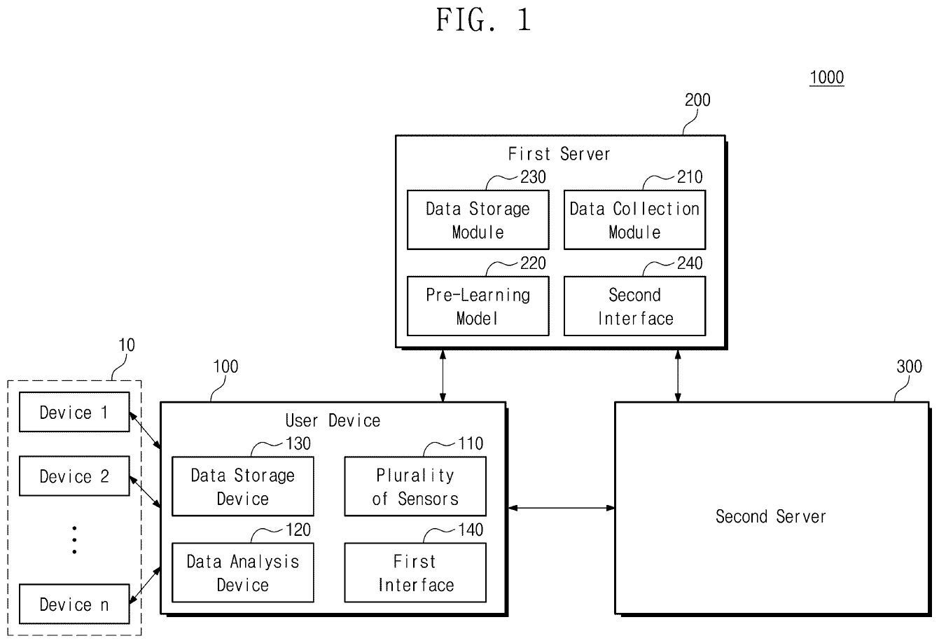

FIG. 1 is a diagram illustrating an electronic device 1000 for generating a multi-persona, according to an embodiment of the present disclosure. Referring to FIG. 1, an electronic device 1000 that generates a multi-persona may include a user device 100, a first server 200, and a second server 300.

The user device 100 may be at least one of a mobile device such as a smartphone, smart watch, and tablet PC (Personal Computer), a computing device such as a laptop computer, computer, peripheral device, and artificial intelligence speaker, and Internet of Things device such as a CCTV, smart lighting, and thermo-hygrometer. However, the user device 100 is not limited thereto.

The user device 100 may include a plurality of applications (not illustrated). The plurality of applications (not illustrated) may collect user data in real time, including GPS (Global Positioning System) data, audio data, video data, lifelogging data, fitness data, health data, journaling data, time management data, SNS (Social Network Service) data, mail data, and message data.

The user device 100 may include a device system (not illustrated). The device system (not illustrated) may collect system data in real time, including charger connection data, earphone and speaker output connection data, network connection data, screen lock time data, screen usage time data, application usage time data, and device usage data.

The user device 100 may include a plurality of sensors 110, a data analysis device 120, a data storage device 130, and a first interface 140.

The plurality of sensors 110 may include an Inertial Measurement Unit (IMU) sensor, an illumination sensor, an atmospheric pressure sensor, a proximity sensor, a fingerprint sensor, a Photoplethysmography (PPG) sensor, and an Electrodermal Activity (EDA) sensor. The plurality of sensors 110 may collect sensor data in real time.

Hereinafter, in the specification, sensor data, user data, and system data are collectively referred to as mobile sensing data or sensing data.

The user device 100 may receive mobile sensing data in real time from a plurality of devices (first device to n-th device).

The data analysis device 120 may receive the mobile sensing data from the plurality of sensors 110 and a plurality of devices (first device to n-th device).

The data analysis device 120 may operate in synchronization with a pre-learning model 220 included in the first server 200, which will be described later. In detail, the data analysis device 120 may analyze the mobile sensing data to generate personal characteristic data and may retrain the pre-learning model 220. A detailed description of the configuration for analyzing the mobile sensing data will be described later.

The data storage device 130 may store personal characteristic data. The data storage device 130 may store the mobile sensing data received from the plurality of sensors 110 and the plurality of devices (the first device to the n-th device).

The data storage device 130 may store not only the mobile sensing data collected in real time but also the mobile sensing data collected in the past. The mobile sensing data collected in the past may be data shared with the plurality of applications (not illustrated) included in the user device 100.

The data storage device 130 may store personal characteristic data. The data storage device 130 may store the personal characteristic data as default parameters. The personal characteristic data may include information about at least one of personality and disposition of the user, feelings and emotional patterns of the user, and life patterns of the user.

The first interface 140 may include a server interface and a user interface. The first interface 140 may provide remote communication between the user device 100 and the first server 200. The first interface 140 may provide wired or wireless communication between the user device 100 and the first server 200.

The first interface 140 may include a device for exchanging information with the user. For example, the first interface 140 may include a display, a speaker, and a touch pad. However, the first interface 140 may further include all devices capable of exchanging information with the user.

The user device 100 may provide default parameters and personal characteristic data to the user through the first interface 140. The user device 100 may receive a user input with respect to default parameters and may output the default parameters and the personal characteristic data as a metaverse persona to the second server 300 through the first interface 140.

The metaverse persona output to the second server 300 may configured to be combined with a digital avatar and a metaverse avatar. The user device 100 may provide the metaverse persona output to the second server 300 to the user through the first interface 140.

The user device 100 may receive the user's mobile sensing data with respect to the metaverse persona. In this case, the mobile sensing data may be input to the data analysis device 120.

The first server 200 may include a data collection module 210, the pre-learning model 220, a data storage module 230, and a second interface 240.

The first server 200 may operate in synchronization with the user device 100. For example, the data analysis device 120 included in the user device 100 may be synchronized with the pre-learning model 220 included in the first server 200 and the analysis parameters included in the pre-learning model 220 to share data.

The data collection module 210 may receive the mobile sensing data from the user device 100 through the first interface 140. Alternatively, the data collection module 210 may additionally collect the mobile sensing data within the first server 200 or from the second server 300. The mobile sensing data collected by the data collection module 210 may be shared with the user device 100 and may be stored in the data storage device 130.

The pre-learning model 220 may be trained to group each of a plurality of personal characteristics for users' mobile sensing data received from a plurality of devices (the first device to the n-th device) 10 based on a convolutional neural network. Additionally, the pre-learning model 220 may be trained to categorize each of the plurality of grouped personal characteristics.

The convolutional neural network may be based on R-CNN, Fast R-CNN, Faster R-CNN, Mask R-CNN, or various similar types of convolutional neural networks, but is not limited to the aforementioned networks.

The pre-learning model 220 may be retrained to output the personal characteristic data when the mobile sensing data is input based on a neural network. In this case, the pre-learning model 220 may be retrained as a personalized analysis model.

The pre-learning model 220 may output a personal characteristic corresponding to the mobile sensing data among a plurality of grouped and categorized personal characteristics. A detailed description of the configuration for outputting personal characteristic data will be described later.

The data storage module 230 may store the personal characteristic data as default parameters. The data storage module 230 may be operated in synchronization with the data storage device 130 included in the user device 100. In this case, the personal characteristic data stored by the data storage module 230 may be synchronized with the personal characteristic data stored in the data storage device 100.

The data storage module 230 may store the mobile sensing data collected by the data collection module 210. In this case, the mobile sensing data may be data collected in the past. The data storage module 230 may store grouped and categorized data for each of a plurality of personal characteristics.

The second interface 240 may include a device interface, a server interface, and a user interface. The second interface 240 may provide remote communication between the first server 200 and the user device 100. The second interface 240 may provide wired or wireless communication between the user device 100 and the first server 200.

The second interface 240 may provide remote communication between the first server 200 and the second server 300. The second interface 240 may provide wired or wireless communication between the first server 200 and the second server 300.

The first server 200 may transmit the mobile sensing data collected through the data collection module 210 to the user device 100 through the second interface 240.

The first server 200 may provide default parameters and personal characteristic data to the user through the second interface 240. The first server 200 may receive user input and may output default parameters and personal characteristic data as a metaverse persona to the second server 300 through the second interface 240.

The metaverse persona output to the second server 300 may configured to be combined with a digital avatar and a metaverse avatar. The first server 200 may provide the metaverse persona output to the second server 300 to the user through the second interface 240.

The first server 200 may receive the user's mobile sensing data with respect to the metaverse persona. In this case, the mobile sensing data may be input to the pre-learning model 220.

The second server 300 may include various metaverse environments. The second server 300 may receive different metaverse personas for each of the metaverse environments.

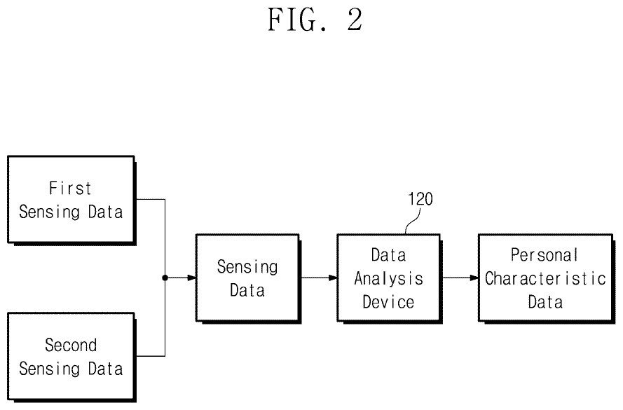

FIG. 2 is a block diagram illustrating how a data analysis device utilizes sensing data. The data analysis device may operate in synchronization with the pre-learning model of the first server. Therefore, among the data analysis device and the pre-learning model, only the data analysis device are described.

Referring to FIGS. 1 and 2, the sensing data input to the data analysis device 120 may include first sensing data collected and stored in the past and second sensing data collected in real time.

In detail, sensing data D3 may be data that reflects both past and present states. The sensing data may be defined by Equation 1 below.

D3=α*D1+(1−α)*D2 [Equation 1]

The sensing data D3 may be defined as the sum of first sensing data D1 and second sensing data D2 multiplied by their respective weights. In operation S120, which will be described later, when a capacity of the first sensing data is less than a capacity of the second sensing data, operation S130 may proceed. In operation S130, which will be described later, when a comparison value of the first sensing data and the second sensing data is less than or equal to a threshold value, operation S140, which will be described later, may proceed. In operation S140, when a data trust value of the first sensing data is greater than a threshold value, operation S150 may proceed. In operation S150, which will be described later, the data analysis device 120 may maintain a weight ‘α’ of the first sensing data.

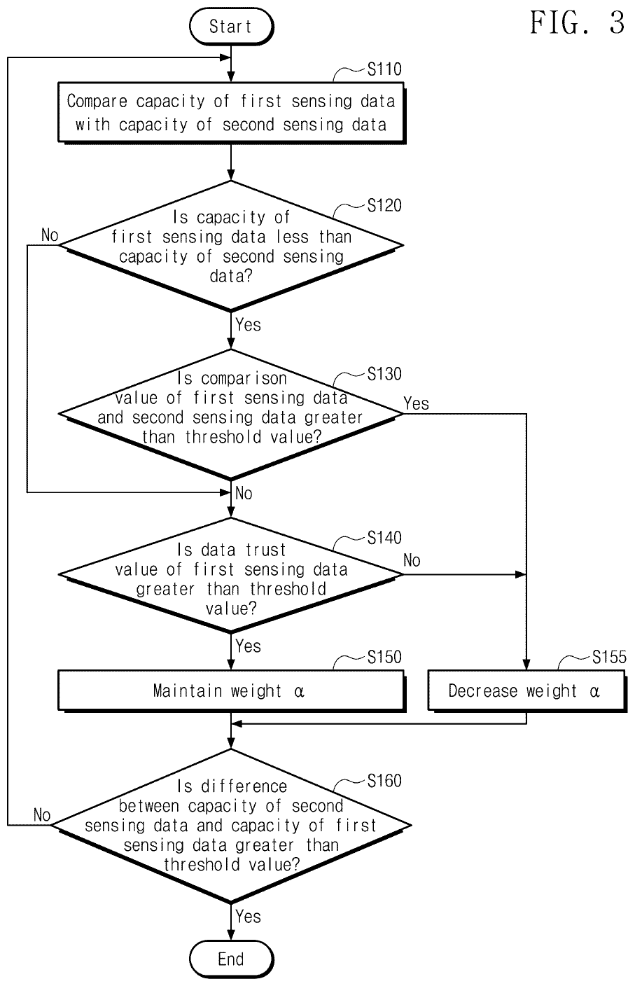

In this case, a weighted average may be reflected. A description of the weight ‘α’ will be described later in FIG. 3.

The data analysis device 120 may vary the weight ‘α’ of the first sensing data D1 and a weight ‘1−α’ of the second sensing data D2. The data analysis device 120 may generate the personal characteristic data by analyzing the sensing data D3. A detailed description of personal characteristic data will be described later.

FIG. 3 is a flowchart for determining sensing data received by a data analysis device. Referring to FIGS. 1 to 3, in operation S110, the data analysis device 120 may compare the capacity of the first sensing data with the capacity of the second sensing data.

In operation S120, when the capacity of the first sensing data is less than the capacity of the second sensing data, operation S130 may proceed. In operation S130, when a comparison value of the first sensing data and the second sensing data is greater than the threshold value, operation S155 may proceed. In operation S155, the data analysis device 120 may decrease the weight ‘α’ of the first sensing data. In this case, the weight ‘1−α’ of the second sensing data may increase. Subsequently, operation S160 may proceed.

In detail, when the amount of first sensing data is less than the capacity of the second sensing data, and when the comparison value of the first sensing data and the second sensing data is greater than the threshold value, the weight ‘α’ of the first sensing data may decrease.

In operation S120, when the capacity of the first sensing data is less than the capacity of the second sensing data, operation S130 may proceed. In operation S130, when the comparison value of the first sensing data and the second sensing data is less than or equal to the threshold value, operation S140 may proceed. In operation S140, when the data trust value of the first sensing data is greater than the threshold value, operation S150 may proceed. In operation S150, the data analysis device 120 may maintain the weight ‘α’ of the first sensing data. Subsequently, operation S160 may proceed.

In detail, when the amount of first sensing data is less than the capacity of the second sensing data, when the comparison value of the first sensing data and the second sensing data is less than or equal to the threshold value, and when the data trust value of the first sensing data is greater than the threshold value, the weight ‘α’ of the first sensing data may be maintained.

In operation S120, when the capacity of the first sensing data is less than the capacity of the second sensing data, operation S130 may proceed. In operation S130, when the comparison value of the first sensing data and the second sensing data is less than or equal to the threshold value, operation S140 may proceed. In operation S140, when the data trust value of the first sensing data is less than or equal to the threshold value, operation S155 may proceed. In operation S155, the data analysis device 120 may decrease the weight ‘α’ of the first sensing data. In this case, the weight ‘1−α’ of the second sensing data may increase. Subsequently, operation S160 may proceed.

In detail, when the amount of first sensing data is less than the capacity of the second sensing data, when the comparison value of the first sensing data and the second sensing data is less than or equal to the threshold value, and when the data trust value of the first sensing data is less than or equal to the threshold value, the weight ‘α’ of the first sensing data may decrease.

In operation S120, when the capacity of the first sensing data is greater than or equal to the capacity of the second sensing data, operation S140 may proceed. In operation S140, when the data trust value of the first sensing data is greater than the threshold value, operation S150 may proceed. In operation S150, the data analysis device 120 may maintain the weight ‘α’ of the first sensing data. Subsequently, operation S160 may proceed.

In detail, when the amount of first sensing data is greater than or equal to the capacity of the second sensing data, and when the data trust value of the first sensing data is greater than the threshold value, the weight ‘α’ of the first sensing data may be maintained.

In operation S120, when the capacity of the first sensing data is greater than or equal to the capacity of the second sensing data, operation S140 may proceed. In operation S140, when the data trust value of the first sensing data is less than or equal to the threshold value, operation S155 may proceed. In operation S155, the data analysis device 120 may decrease the weight ‘α’ of the first sensing data. In this case, the weight ‘1−α’ of the second sensing data may increase. Subsequently, operation S160 may proceed.

In detail, when the amount of first sensing data is greater than or equal to the capacity of the second sensing data, and when the data trust value of the first sensing data is less than the threshold value, the weight ‘α’ of the first sensing data may decrease.

The comparison value of the first sensing data and the second sensing data may be determined depending on the degree of similarity between the data pattern of the first sensing data and the data pattern of the second sensing data.

The data trust value may be determined by at least one of information about the source from which the data is acquired and the time at which the data is acquired. For example, the data trust value of first sensing data collected from applications included in the user device 100 may be greater than that of first sensing data collected from external applications not included in the user device 100.

As an example, by comparing the time when the user device 100 acquires the first sensing data with the current time, as the time interval is shorter, the data reliability value of the first sensing data may be greater.

In operation S160, the data analysis device 120 may compare a difference between the capacity of the second sensing data and the capacity of the first sensing data with a threshold value.

When the difference between the capacity of the second sensing data and the capacity of the first sensing data is greater than the threshold value, the procedure is terminated. When the difference between the capacity of the second sensing data and the capacity of the first sensing data is less than or equal to the threshold value, operations S110 to S160 may be repeatedly performed.

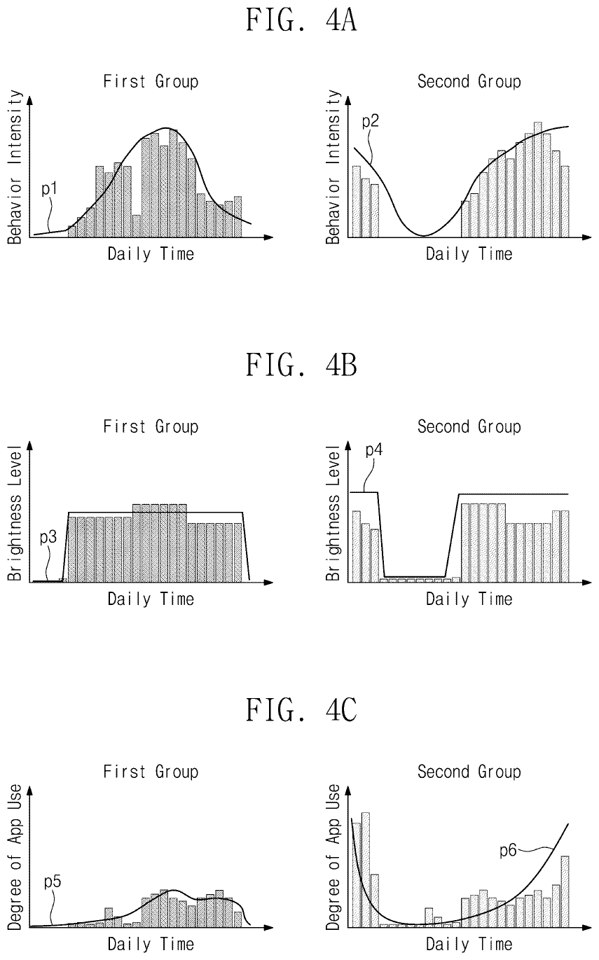

FIG. 4A is a diagram related to a first embodiment of a life pattern analysis result generated by a data analysis device. By way of example, in FIG. 4A, personal characteristic data is illustrated in the form of a two-dimensional graph. In FIG. 4A, a horizontal axis represents daily time, and a vertical axis represents behavioral intensity.

In FIG. 4A, a bar graph represents the behavior intensity per unit time during one day, and a line graph represents the life pattern according to the change in the behavior intensity during one day.

Referring to FIGS. 1 and 4A, the data analysis device 120 may generate personal characteristic data about the behavior intensity in the user's life pattern based on sensing data about the user's movements.

For example, when the behavior intensity expressed as the line graph appears as a first pattern p1 during one day, the data analysis device 120 may classify the user into a first group. The first pattern p1 may be a two-dimensional curve convex upward. In this case, the first group may be an early-bird group.

For example, when the behavior intensity expressed in the line graph appears as a second pattern p2, the data analysis device 120 may classify the user into a second group. The second pattern p2 may be a two-dimensional curve convex downward. In this case, the second group may be a Night-Owl group.

FIG. 4B is a diagram related to a second embodiment of a life pattern analysis result generated by a data analysis device. By way of example, in FIG. 4B, personal characteristic data is illustrated in the form of a two-dimensional graph. In FIG. 4B, a horizontal axis represents daily time, and a vertical axis represents brightness level.

In FIG. 4B, a bar graph represents the brightness level per unit time during one day, and a line graph represents the life pattern according to the change in brightness level during one day. Additional descriptions of similar contents to those of FIG. 4A will be omitted to avoid redundancy.

Referring to FIGS. 1 and 4B, the data analysis device 120 may generate personal characteristic data regarding the level of brightness in the user's lifestyle pattern based on sensing data about the illuminance of the user's surrounding environment.

For example, when the level of brightness expressed in the line graph appears as a third pattern p3, the data analysis device 120 may classify the user into the first group. The third pattern p3 may be a square wave convex upward.

For example, when the level of brightness expressed in the line graph appears as a fourth pattern p4, the data analysis device 120 may classify the user into the second group. The fourth pattern p4 may be a square wave convex downward.

FIG. 4C is a diagram related to a third embodiment of a life pattern analysis result generated by a data analysis device. By way of example, in FIG. 4C, personal characteristic data is illustrated in the form of a two-dimensional graph. In FIG. 4C, a horizontal axis represents daily time, and a vertical axis represents the degree of app use.

In FIG. 4C, a bar graph represents the degree of app use per unit time during one day, and a line graph represents life patterns according to changes in the degree of app use during one day. Additional descriptions of similar contents to those of FIG. 4A will be omitted to avoid redundancy.

Referring to FIGS. 1 and 4C, the data analysis device 120 may generate personal characteristic data regarding the degree of app use among life patterns based on sensing data about the user's application use.

For example, when the degree of app use appears as a fifth pattern p5, the data analysis device 120 may classify the user into the first group. The fifth pattern p5 may be a curve convex upward.

For example, when the degree of app use appears as a sixth pattern p6, the data analysis device 120 may classify the user into the second group. The sixth pattern p6 may be a curve convex downward.

FIGS. 4A to 4C illustrate personal characteristic data regarding behavior intensity, brightness degree, and app usage degree among lifestyle patterns. However, without being limited thereto, personal characteristic data may be data in which deviation, amount of change, similarity, periodicity, and pattern within a time interval are calculated for various aspects observed during daily life, such as location and location changes, and environment and environmental changes.

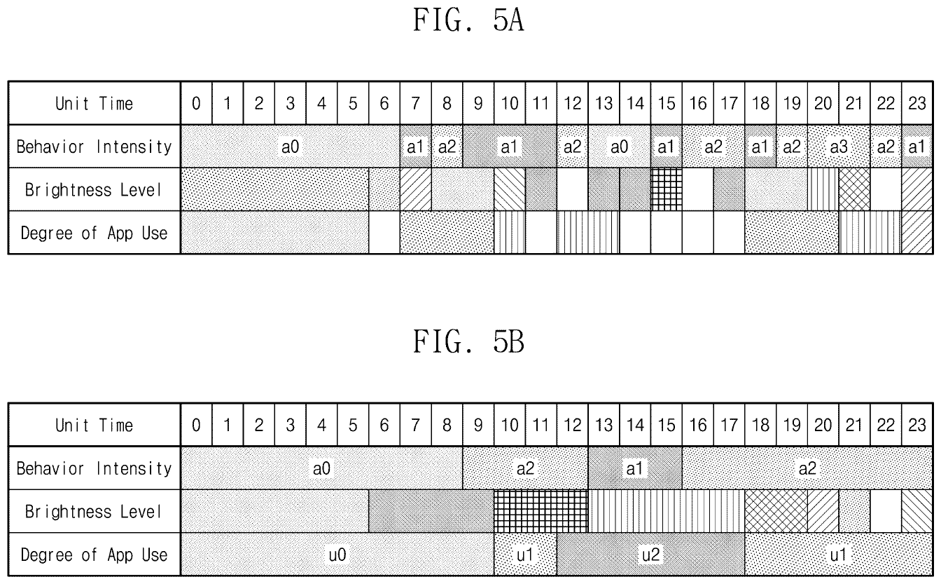

FIG. 5A is a diagram related to a first embodiment illustrating a correlation between various sensing data. In FIG. 5A, 0 to 23 represent unit time. As an example, in FIG. 5A, mobile sensing data may include first to third data.

The first data may be sensing data that may indicate the behavior intensity among personal characteristics. For example, the first data may be sensing data about the user's movement collected from an IMU sensor among the plurality of sensors 110.

The second data may be sensing data that may indicate the level of brightness among personal characteristics. For example, the second data may be sensing data about the user's surrounding environment collected from an illumination sensor among the plurality of sensors 110.

The third data may be sensing data that may indicate the degree of app use among personal characteristics. For example, the third data may be sensing data about application usage time collected from the device system.

In FIG. 5A, in the first to seventh, fourteenth, and fifteenth unit times (0 to 6, 13, and 14), the user's behavior intensity may be at a first level a0. At 8th, 10th to 12th, 16th, 19th, and 24th unit times (7, 9 to 11, 15, 18, and 23), the user's behavior intensity may be at a second level a1. At 9th, 13th, 17th, 18th, 20th, and 23rd unit times (8, 12, 16, 17, 19, and 22), the user's behavior intensity may be at a third level a2. At 21st and 22nd unit times (20 and 21), the user's behavior intensity may be at a fourth level a3.

For example, the first level a0 may be lower than the second to fourth levels a1 to a3. The second level a1 may be higher than the first level a0 and lower than the third and fourth levels a2 and a3. The third level a2 may be higher than the first and second levels a0 and a1 and lower than the fourth level a3. The fourth level a3 may be higher than the first to third levels a0 to a2.

Although not illustrated, as in the above description, for each unit of time, the brightness of the user's surrounding environment and the user's app use may be at different levels in all or some of them.

Referring to FIGS. 1 and 5A, the first to third data received as sensing data by the data analysis device 120 may have a low correlation with each other. In this case, the data analysis device 120 may receive first to third data respectively and may generate personal characteristic data.

FIG. 5B is a diagram related to a second embodiment illustrating a correlation between various sensing data. In FIG. 5B, 0 to 23 represent unit time. As an example, in FIG. 5B, mobile sensing data may include first to third data. Additional descriptions of similar contents to those of FIG. 5A will be omitted to avoid redundancy.

In FIG. 5B, at first to ninth unit times (0 to 8), the user's behavior intensity may be at the first level a0. At 14th to 16th unit times (13 to 15), the user's behavior intensity may be at the second level a1. At 10th to 13th and 17th to 24th unit times (9 to 12 and 16 to 23), the user's behavior intensity may be at the third level a2.

In FIG. 5B, at the first to tenth unit times (0 to 9), the user's app usage degree may be at a first level u0. At the 11th, 12th, and 19th to 24th unit times (10, 11, 18 to 23), the user's app usage degree may be at a second level u1. At the 13th to 18th unit times (12 to 17), the user's app usage degree may be at a third level u2.

Referring to FIGS. 1 and 5B, the first data and third data received as sensing data by the data analysis device 120 may have a high correlation with each other.

For example, at the 1st to 9th unit times (0 to 8), the 11th and 12th unit times (10 to 11), the 14th to 16th unit times (13 to 15), and the 19th to 24th unit times (18 to 23), there may be a high correlation between the user's behavior intensity and the user's app use degree. In this case, the data analysis device 120 may receive the first data and the third data as one vector.

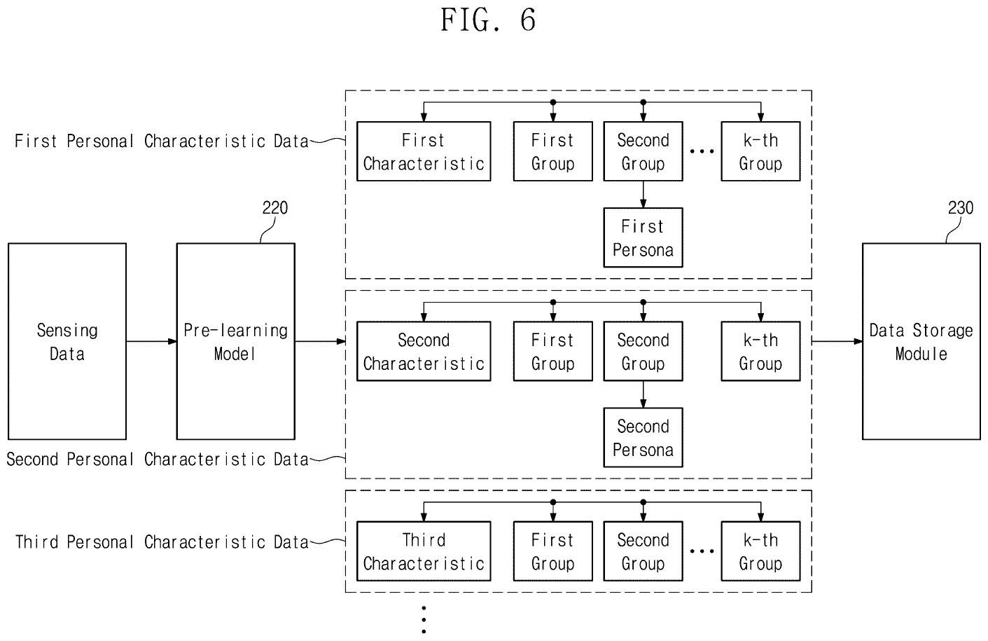

FIG. 6 is a block diagram illustrating how a pre-learning model analyzes sensing data and groups personal characteristic data. Referring to FIGS. 1 to 6, the pre-learning model 220 may receive mobile sensing data from users and may be trained to group a plurality of personal characteristics.

In FIG. 6, the pre-learning model 220 may classify each of a plurality of personal characteristics (first characteristic to third characteristic) into a plurality of groups (first group to k-th group, for example, ‘k’ is a natural number of 3 or more). However, the personal characteristics output from the pre-learning model 220 are not limited thereto.

subsequently, the pre-learning model 220 may receive the sensing data of the user. In this case, the pre-learning model 220 may output first to third personal characteristic data based on the sensing data, and may update analysis parameters based on each of the output first to third personal characteristic data.

The pre-learning model 220 may output first personal characteristic data based on the sensing data. A second group belonging to the first characteristic in the first personal characteristic data may be stored in the data storage module 230 as the first persona, which is a default parameter. The first persona may be the user's representative persona.

The pre-learning model 220 may output second personal characteristic data based on the sensing data. A second group belonging to the second characteristic in the second personal characteristic data may be stored in the data storage module 230 as the second persona, which is the default parameter. The second persona may be the user's candidate persona. The first persona and the second persona may be defined as the multi-persona.

The pre-learning model 220 may output third personal characteristic data based on the sensing data.

The data storage module 230 may provide default parameters, which are the first persona and the second persona, to the user through the second interface 240.

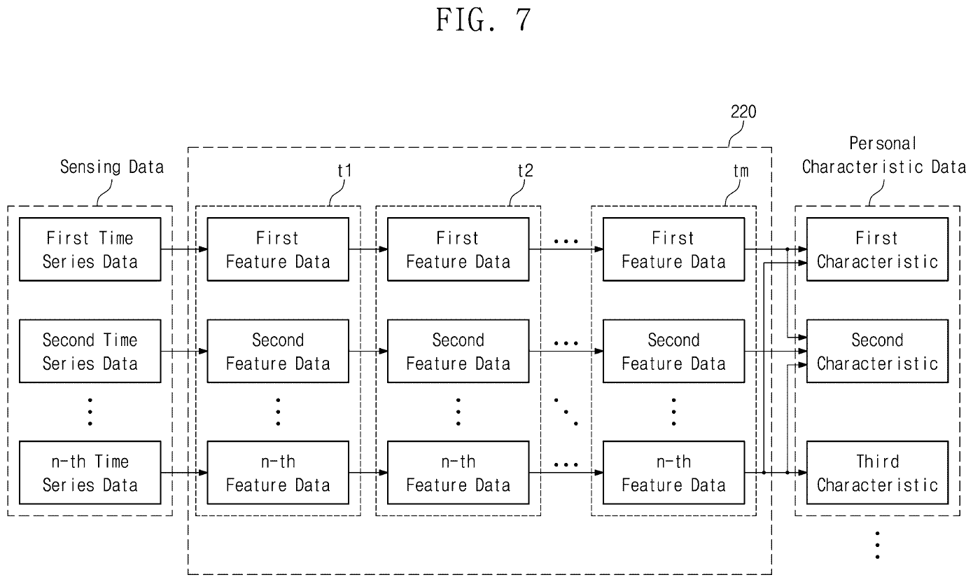

FIG. 7 is a block diagram illustrating how a pre-learning model performs hierarchical unit time analysis on sensing data and outputs personal characteristics. In FIG. 7, t1 to tm may mean unit time.

Referring to FIGS. 1 and 7, the pre-learning model 220 may receive sensing data. The sensing data may include first to n-th time series data.

The pre-learning model 220 may hierarchically analyze first time series data as first feature data. For example, the pre-learning model 220 may output the first feature data during the first unit time t1 based on the first time series data, and may newly output data accumulated during the first and second unit times t1 and t2 as the first feature data, based on an output of the first feature data during the first unit time t1. In this way, data accumulated during the first to m-th unit times t1 to tm may be newly output as the first feature data.

The pre-learning model 220 may hierarchically analyze the second time series data as second feature data. For example, the pre-learning model 220 may output the second feature data during the first unit time t1 based on the second time series data, and may newly output data accumulated during the first and second unit times t1 and t2 as the second feature data, based on an output of the second feature data during the first unit time t1. In this way, data accumulated during the first to m-th unit times t1 to tm may be newly output as the second feature data.

In this way, the pre-learning model 220 may hierarchically analyze n-th time series data as n-th feature data. For example, the pre-learning model 220 may output the n-th feature data during the first unit time t1 based on the n-th time series data, and may newly output data accumulated during first and second unit times t1 and t2 as the n-th feature data, based on an output of the n-th feature data during the first unit time t1. In this way, data accumulated during the first to m-th unit times t1 to tm may be newly output as the n-th feature data.

In detail, the pre-learning model 220 may output each data accumulated for the first to m-th unit times t1 to tm as the first to n-th feature data to hierarchically analyze the first to n-th time series data.

For example, the first unit time t1 may be shorter than other unit times. The second unit time t2 may be longer than the first unit time t1 and may be shorter than the remaining unit times. The m-th unit time tm may be longer than other unit times.

Each of the first to m-th unit times t1 to tm may be one of general time units such as seconds, minutes, hours, days, weeks, months, quarters, and years. Each of the first to m-th unit times t1 to tm may be one of semantically distinct time units such as breakfast, lunch, dinner, and work time.

The pre-learning model 220 may output personal characteristic data based on at least one of the first to n-th time series data. The personal characteristic data may include first to third characteristics. However, the personal characteristic data is not limited to thereto.

For example, the first characteristic may be output based on hierarchical analysis results of each of the first time series data and the n-th time series data. The second characteristic may be output based on hierarchical analysis results of each of the first time series data, second time series data, and n-th time series data. The third characteristic may be output based on the hierarchical analysis result of the n-th time series data.

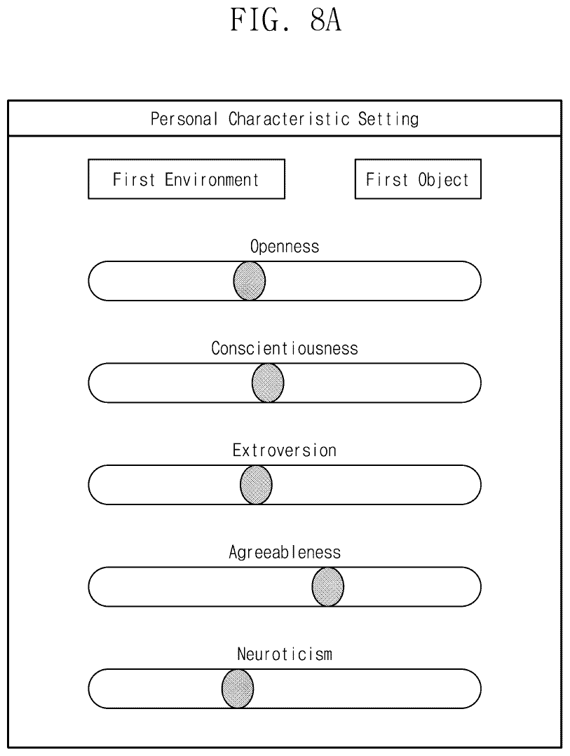

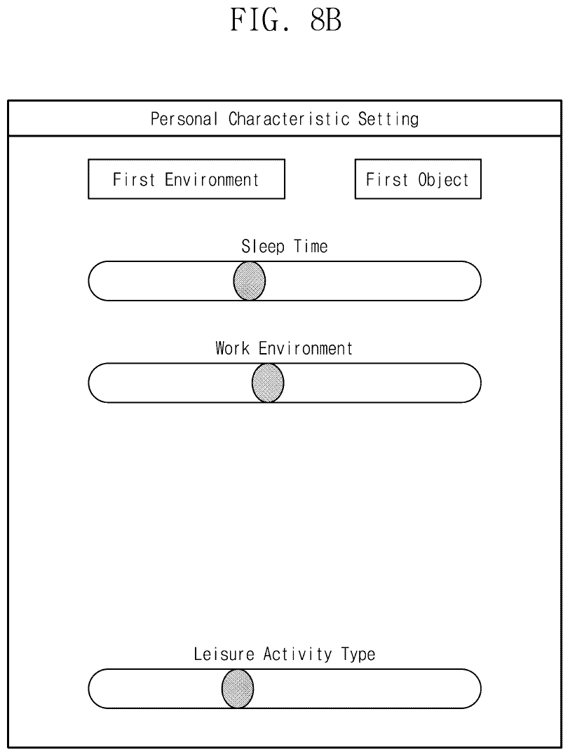

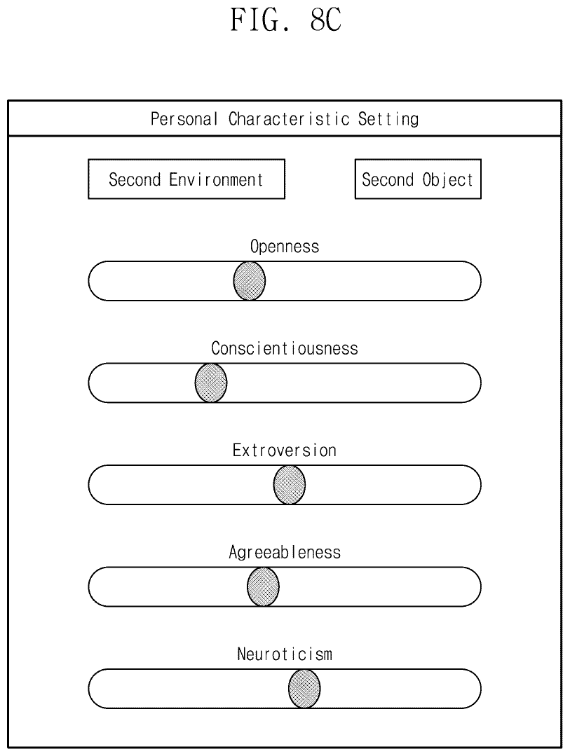

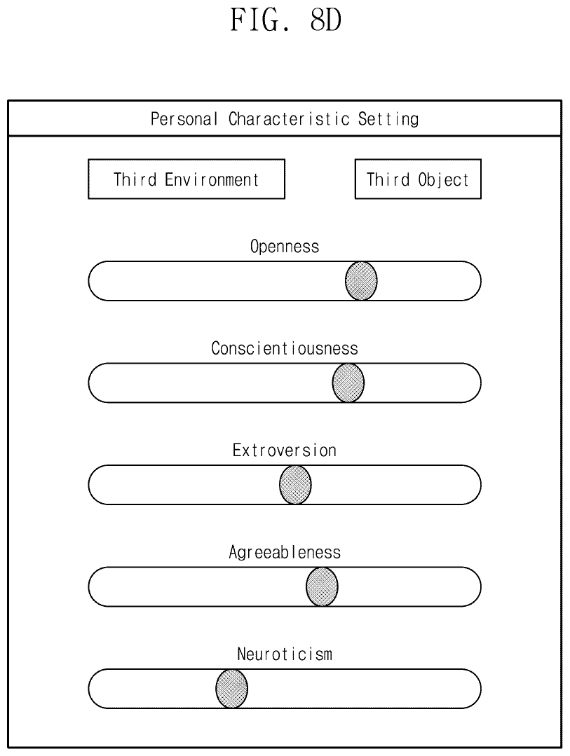

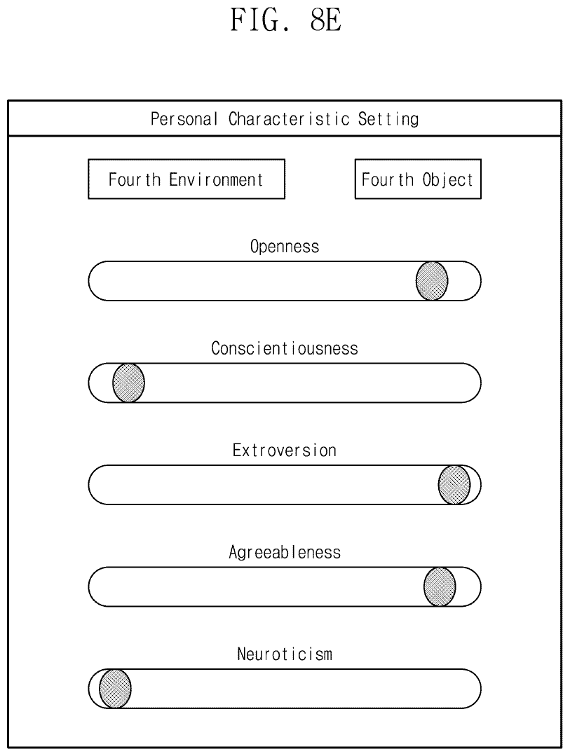

FIG. 8A is a diagram related to a first embodiment of personal characteristic settings provided by a user device. As an example, in FIG. 8A, among the user's personal characteristics, personality includes openness, conscientiousness, extroversion, agreeableness, and neuroticism. In FIG. 8A, the categories corresponding to each characteristic are provided for selection in a slider format. However, the personality trait and category selection method are not limited thereto.

Referring to FIGS. 1 to 8A, the user device 100 may provide a first environment, a first object, and a first metaverse persona including personality characteristics among personal characteristics to the user through the first interface 140. The first environment may be the default metaverse environment, and the first object may be the corresponding user of the user device 100.

In FIG. 8A, sliders displayed for each of openness, conscientiousness, extraversion, agreeableness, and neuroticism may be presented to the user as default parameters.

The user device 100 may receive user input from the user. The user input may include modifications and selections with respect to the first environment, first object, and default parameters for personality characteristics.

FIG. 8B is a diagram related to a second embodiment of personal characteristic settings provided by a user device. As an example, in FIG. 8B, among the user's personal characteristics, the life pattern includes sleep time, work environment, and leisure activity type.

In FIG. 8B, the categories corresponding to each characteristic are provided for selection in a slider format. However, the lifestyle pattern characteristics and category selection method are not limited thereto. Additional descriptions of similar contents to those of FIG. 8A will be omitted to avoid redundancy.

Referring to FIGS. 1 to 8B, the user device 100 may provide a first environment, a first object, and a second metaverse persona including lifestyle pattern characteristics among personal characteristics to the user through the first interface 140. In FIG. 8B, sliders displayed for each of sleep time, work environment, and leisure activity type may be provided to the user as default parameters.

The user device 100 may receive user input from the user. The user input may include modifications and selections with respect to the first environment, first object, and default parameters for lifestyle pattern characteristics.

FIG. 8C is a diagram related to a third embodiment of personal characteristic settings provided by a user device. As an example, in FIG. 8C, among the user's personal characteristics, personality includes openness, conscientiousness, extroversion, agreeableness, and neuroticism.

In FIG. 8C, the categories corresponding to each characteristic are provided for selection in a slider format. However, the personality trait and category selection method are not limited thereto. Additional descriptions of similar contents to those of FIG. 8A will be omitted to avoid redundancy.

Referring to FIGS. 1 to 8C, the user device 100 may provide a second environment, a second object, and a third metaverse persona including personality characteristics among personal characteristics to the user through the first interface 140. The second environment may be a game environment, and the second object may be a professional gamer.

In FIG. 8C, the user device 100 may provide the user with default parameters for a third metaverse persona that is different from the first metaverse persona with respect to a second environment that is different from the first environment.

The user device 100 may receive user input from the user. User input may include modifications and selections to default parameters for the second environment, second object, and personality characteristics.

FIG. 8D is a diagram related to a fourth embodiment of personal characteristic settings provided by a user device. As an example, in FIG. 8D, among the user's personal characteristics, personality includes openness, conscientiousness, extroversion, agreeableness, and neuroticism.

In FIG. 8D, the categories corresponding to each characteristic are provided for selection in a slider format. However, the personality trait and category selection method are not limited thereto. Additional descriptions of similar contents to those of FIG. 8A will be omitted to avoid redundancy.

Referring to FIGS. 1 to 8D, the user device 100 may provide a third environment, a third object, and a fourth metaverse persona including personality characteristics among personal characteristics to the user through the first interface 140. The third environment may be a business environment, and the third object may be an expert in the relevant field.

In FIG. 8D, the user device 100 may provide the user with default parameters for a fourth metaverse persona that is different from the third metaverse persona with respect to a third environment that is different from the second environment.

The user device 100 may receive user input from the user. The user input may include modifications and selections with respect to the third environment, third object, and default parameters for personality characteristics.

FIG. 8E is a diagram related to a fifth embodiment of personal characteristic settings provided by a user device. As an example, in FIG. 8E, among the user's personal characteristics, personality includes openness, conscientiousness, extroversion, agreeableness, and neuroticism.

In FIG. 8E, the categories corresponding to each characteristic are provided for selection in a slider format. However, the personality trait and category selection method are not limited thereto. Additional descriptions of similar contents to those of FIG. 8A will be omitted to avoid redundancy.

Referring to FIGS. 1 to 8E, the user device 100 may provide a fourth environment, a fourth object, and a fifth metaverse persona including personality characteristics among personal characteristics to the user through the first interface 140. The fourth environment may be a broadcasting station environment, and the fourth object may be a celebrity.

In FIG. 8E, the user device 100 may provide the user with default parameters for a fifth metaverse persona that is different from the fourth metaverse persona with respect to a fourth environment that is different from the third environment.

The user device 100 may receive user input from the user. The user input may include modifications and selections with respect to the fourth environment, fourth object, and default parameters for personality characteristics.

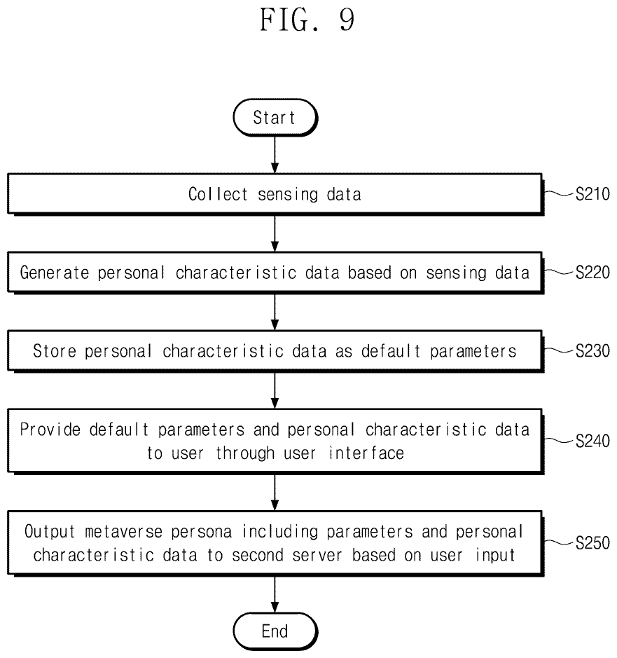

FIG. 9 is a flowchart of an operation method for generating a multi-persona by an electronic device, according to the present disclosure. Referring to FIGS. 1 to 9, in operation S210, the plurality of sensors 110 or the data collection module 210 included in the electronic device 1000 may collect sensing data.

In operation S220, the electronic device 1000 may generate personal characteristic data based on the sensing data. The electronic device 1000 may include the pre-learning model 220. The sensing data may be input as an input to the pre-learning model 220, and the personal characteristic data may be output as an output of the pre-learning model 220.

In operation S230, the electronic device 1000 may store the personal characteristic data as default parameters. The default parameters may include a parameter for the first persona and a parameter for the second persona.

In operation S240, the electronic device 1000 may provide the default parameters and the personal characteristic data to the user through the first and second interfaces 140 and 240. The electronic device 1000 may provide users with different metaverse personas for each of the various metaverse environments.

In operation S250, the electronic device 1000 may output a metaverse persona including parameters and the personal characteristic data to the second server 300 based on the user input. The electronic device 1000 may output the multi-metaverse persona to various metaverse environments included in the second server 300.

According to an embodiment of the present disclosure, an electronic device and its operating method may analyze individual characteristics and may form a multi-persona based on sensing data through a mobile device. In addition, according to an embodiment of the present disclosure, the electronic device may improve the user's experience by enabling addition and modification of various personal characteristics with respect to the metaverse persona.

The above descriptions are specific embodiments for carrying out the present disclosure. Embodiments in which a design is changed simply or which are easily changed may be included in the present disclosure as well as an embodiment described above. In addition, technologies that are easily changed and implemented by using the above embodiments may be included in the present disclosure. While the present disclosure has been described with reference to embodiments thereof, it will be apparent to those of ordinary skill in the art that various changes and modifications may be made thereto without departing from the spirit and scope of the present disclosure as set forth in the following claims.

What is claimed is:

1. A user device comprising:
a plurality of sensors configured to collect sensing data;
a data analysis device configured to receive the sensing data from a first server or the plurality of sensors to generate personal characteristic data; and
a data storage device configured to store the personal characteristic data as default parameters, and
wherein the data storage device provides the default parameters and the personal characteristic data to a user through an interface, and
wherein the data storage device outputs parameters and the personal characteristic data to a second server based on a user input.

2. The user device of claim 1, wherein the personal characteristic data includes information about at least one of personality and disposition of the user, feelings and emotional patterns of the user, and life patterns of the user.

3. The user device of claim 1, wherein the data analysis device receives the sensing data in synchronization with a pre-learning model included in the first server and generates the personal characteristic data.

4. The user device of claim 3, wherein the pre-learning model is trained to group each of a plurality of personal characteristics by receiving users' sensing information from the data storage device before the data analysis device receives the sensing data, and
wherein, when the sensing data is received, the data analysis device outputs a personal characteristic corresponding to the sensing data among the plurality of grouped personal characteristics, and retrains the pre-learning model.

5. The user device of claim 4, wherein the data analysis device outputs first personal characteristic data including a first persona based on the sensing data, and
wherein the data analysis device outputs second personal characteristic data including a second persona based on the sensing data.

6. The user device of claim 5, wherein the data storage device stores the first personal characteristic data and the second personal characteristic data as the default parameters,
the second server includes a first metaverse environment and a second metaverse environment,
a parameter associated with the first personal characteristic data among the parameters is output to the first metaverse environment as the first persona, and
a parameter associated with the second personal characteristic data among the parameters is output to the second metaverse environment as the second persona.

7. The user device of claim 1, wherein the sensing data includes:
first data stored in the data storage device in advance or received from the first server; and
second data collected in real time from the plurality of sensors, and
wherein the data analysis device receives the first data and the second data by varying a first weight of the first data and a second weight of the second data.

8. The user device of claim 7, wherein, when a capacity of the first data is less than a capacity of the second data, and a comparison value of the first data and the second data is greater than a threshold value,
the data analysis device receives the first data by decreasing the first weight, and
the data analysis device receives the second data by increasing the second weight.

9. The user device of claim 7, wherein, when a capacity of the first data is greater than a capacity of the second data,
the data analysis device decreases or increases the first weight depending on whether a data trust value of the first data exceeds a threshold value.

10. The user device of claim 7, wherein, when a capacity of the first data is less than a capacity of the second data, and a comparison value of the first data and the second data is less than or equal to a threshold value,
the data analysis device decreases or increases the first weight depending on whether a data trust value of the first data exceeds a threshold value.

11. The user device of claim 1, wherein the sensing data includes:
first data including information on a behavior intensity of the user for each unit of time; and
second data including information on an app use degree of the user for the each unit of time, and
wherein the data analysis device receives the first data and the second data as one vector based on a correlation between the first data and the second data.

12. The user device of claim 1, wherein the sensing data includes first to n-th time series data, and
wherein the data analysis device performs a first analysis on the first to n-th time series data for a first time,
the data analysis device performs a second analysis on the first to n-th time series data for a second time after the first time, and
the data analysis device performs a third analysis on the first to n-th time series data for a third time after the second time, and
wherein the personal characteristic data is generated based on at least one of the first to n-th time series data analyzed during the third time,
the second analysis is performed based on a result of the first analysis,
the third analysis is performed based on a result of the second analysis,
the second time is longer than the first time, and
the third time is longer than the second time.

13. A method of operating an electronic device including a user device and a first server, the method comprising:
collecting, by the user device, sensing data;
generating, by the first server, personal characteristic data based on the sensing data received from the user device;
storing, by the first server, the personal characteristics data as default parameters;
providing, by the user device, the default parameters and the personal characteristic data to a user through an interface; and
outputting, by the user device, parameters and the personal characteristic data to a second server based on a user input.

14. The method of claim 13, wherein the generating of the personal characteristic data includes analyzing, by the user device, the sensing data in synchronization with a pre-learning model included in the first server.

15. The method of claim 14, wherein the pre-learning model is trained to group each of a plurality of personal characteristics by receiving users' sensing information before analyzing the sensing data, and
wherein the analyzing of the sensing data includes outputting, by the user device, a personal characteristic corresponding to the sensing data among the plurality of grouped personal characteristics.

16. The method of claim 15, wherein the outputting of the personal characteristic includes:
outputting, by the user device, first personal characteristic data including a first persona based on the sensing data, and
outputting, by the user device, second personal characteristic data including a second persona based on the sensing data.

17. The method of claim 16, wherein the second server includes a first metaverse environment and a second metaverse environment, and
further comprising: storing, by the user device, the first personal characteristic data and the second personal characteristic data as the default parameters;
outputting, by the user device, a parameter associated with the first personal characteristic data among the parameters to the first metaverse environment as the first persona, and
outputting, by the user device, a parameter associated with the second personal characteristic data among the parameters to the second metaverse environment as the second persona.

18. The method of claim 13, wherein the sensing data includes:
first data stored in the user device in advance or received from the first server; and
second data collected by the user device in real time, and
wherein the generating of the personal characteristic data includes:
synchronizing, by the user device, with a pre-learning model included in the first server; and
analyzing, by the user device, the first data and the second data by varying a first weight of the first data and a second weight of the second data.

19. The method of claim 13, wherein the sensing data includes:
first data including information on a behavior intensity of the user for each unit of time; and
second data including information on an app use degree of the user for the each unit of time, and
wherein the generating of the personal characteristic data includes analyzing, by the user device, the first data and the second data as one vector based on a correlation between the first data and the second data.

20. The method of claim 13, wherein the sensing data includes first to n-th time series data, and
wherein the generating of the personal characteristic data includes:
performing, by the user device, a first analysis on the first to n-th time series data for a first time;
performing, by the user device, a second analysis on the first to n-th time series data for a second time after the first time, and
performing, by the user device, a third analysis on the first to n-th time series data for a third time after the second time, and
wherein the personal characteristic data is generated based on at least one of the first to n-th time series data analyzed during the third time,
the second analysis is performed based on a result of the first analysis,
the third analysis is performed based on a result of the second analysis,
the second time is longer than the first time, and
the third time is longer than the second time.
